(19) United States
(12) Patent Application Publication
(10) Pub. No.: US 2004/0225367 A1
(43) Pub. Date: Nov. 11, 2004

(54) Hip-joint and shoulder-joint endoprosthesis

(75) Inventors: Wilfried Glien, Bad Kosterlausnitz (DE); Frank Dallmann, Schmolln (DE); Dirk Salomon, Jonaswalde (DE); Lars Kappel, Triptis (DE); Thomas Oberbach, Reichenbach (DE); Thomas Katzer, Hermsdorf (DE)

Correspondence Address: GRAYBEAL, JACKSON, HALEY LLP, 155 - 108TH AVENUE NE, SUITE 350, BELLEVUE, WA, 98004-5901, US

(73) Assignee: Mathys Orthopadie GmbH

(21) Appl. No.: 10/867,846

(22) Filed: Jun. 14, 2004

Related U.S. Application Data
(63) Continuation-in-part of application No. PCT/EP02/14214, filed on Dec. 13, 2002.

(30) Foreign Application Priority Data
Dec. 14, 2001 (DE) 201 20 241.7

(51) Int. Cl. A61F 2/40; A61F 2/36

(52) U.S. Cl. 623/19.14; 623/23.26; 623/23.35

(57) ABSTRACT

The hip-or shoulder-joint endoprosthesis has a stem (10) and a ball head (20) that has an arched top side (21) and a closed underside (22). The longitudinal axis of the stem (10) forms an angle of at most 30° with the axis of the ball head (20). The stem (10) tapers with an overall aperture angle in the range of 5 to 30°. The ball head (20) has the shape of a sphere section of less than a hemisphere. The length of the stem (10) is less than the diameter of the ball head (20). The longitudinal axis of the stem (10) can coincide with the axis of the ball head (20).

### CROSS-REFERENCE TO RELATED APPLICATION

This application is a continuation-in-part of the PCT Application No. PCT/EP02/14214, filed 13 Dec. 2002, which is herein incorporated by reference, that claims priority from German Patent Application No. 201 20 241.7, filed 14 Dec. 2001, herein incorporated by reference. This application claims the benefit of the filing date of the PCT Application under 35 USC §120; and claims priority from the German Patent Application.

### FIELD OF THE INVENTION

The invention relates to a hip-and shoulder-joint endoprosthesis with a stem and a ball head that is in one piece with the stem or can be attached to the stem. The ball head has an arched top side and a closed underside. The stem and the ball head each have a longitudinal axis and the axis of the ball head runs through the centre of the ball and approximately perpendicular to the underside of the ball.

### BACKGROUND

Conventional endoprostheses are anchored in intramedullary manner via a stem rotated at a particular angle (e.g. 135°) to the head axis, the stem being 10 to 20 cm long and being inserted into the marrow cavity of the bone. Depending on the design of the prosthesis and whether a cement-free or cemented implantation is carried out, an overspecification in the anchoring mechanism can result. If this prosthesis is supported in intramedullary manner by its distal tip, the natural force transmission is diverted away from the proximal corticalis in the ball region into the intramedullary stem region. As a result bone reforms, i.e. bone grows around the distal prosthesis end and bone degenerates in the boundary region between ball head and epiphysis (proximal bone). This is often cited as the cause of loosening. There is also a risk of a diaphyseal stem fracture in the region of the distal prosthesis end either due to a sudden overloading when the prosthesis is distally supported or, when the prosthesis is proximally supported, as a result of bone degradation in the distal prosthesis region.

The reproduction of the anatomical starting situation is made difficult by significant variations in inclination and retrotorsion of the ball head relative to the stem. Although the prosthesis head unavoidably aligns itself in the diaphysis on implantation, in the case of prostheses with fixed inclination and retrotorsion, the ball head often does not lie flat on the resection plane, even despite costly instrumentation. The resultant inhomogeneous force transmission leads to bone reformation and ultimately to loosening. More recent generations of prostheses, in particular in shoulder prosthetics, enable named parameters to be set, albeit at a high technical cost.

To solve this problem it is already known to develop the stem much shorter and not to insert it into the marrow cavity of the bone, but to use a short stem and to align this parallel to the axis of the ball head. In the case of such a hip femoral neck endoprosthesis known from DE 198 34 277 C2, alongside the support on the resection plane, an axial guiding and fixing is additionally provided via a lateral cortical opening. The anchoring is achieved via a fixing at both ends of the stem.

U.S. Pat. No. 4,042,980 shows a humeral component with short stem in which the ball head can be hollow or closed. The stem has circular ribs.

DE 37 07 518 A1 shows a prosthesis with a plurality of anchoring posts that run at different curvatures from the resection plane in the direction of the stem.

DE 196 13 078 A1 describes an anchoring technique by means of a cage structure into the interstices of which spongiosa can grow. This structure or the distally developed stem is likewise rotated starting from the ball axis towards the stem axis and extends into the diaphyseal stem region.

DE 42 20 217 A1 describes a prosthesis for replacing the joint surface, in particular the cartilage layer. According to this the ball is not developed solid and does not close with a smooth surface but is hollowed out cap-shaped inside. There is the risk of an osteolysis with such ball prostheses.

The Rozing-type shoulder endoprosthesis placed on the market by ESKA Implants GmbH & Co., which is modularly constructed and in the shortest extension stage is designed for a purely proximal anchoring, is also known. Here however the stem axis is rotated by a fixed inclination angle vis-à-vis the ball axis in order to permit an intramedullary anchoring in longer extension stages. This brings with it the above-mentioned overspecification upon anchoring as in the case of conventional long-stemmed prostheses.

### SUMMARY OF THE INVENTION

The objective of the present invention is a hip-or shoulder-joint endoprosthesis that can be positioned exactly and permanently fixed with simple means.

This objective is solved according to the invention in that the longitudinal axis of the stem forms an angle of at most 30° with the axis of the ball head, the cone angle of the stem is 5° to 30° and the ball head has the shape of a sphere section of less than a hemisphere, and the length of the stem is less than the diameter of the ball head.

The cone angle of the stem is preferably approximately 20°.

The short-stemmed prosthesis of the invention is anchored exclusively in the epiphysis, the stem axis being rotated through the ball centre by at most 30°, preferably by at most 20°, vis-à-vis the axis. In a particularly preferred version, the stem axis coincides with the axis of the ball head. As a result an overspecification (redundant fixation) upon anchoring is avoided and force transmission via the cortical edge of the resection plane and also by means of press fit in the spongiosa is ensured. To avoid a lateral perforation, the stem is very short, preferably shorter than the diameter of the ball head.

Ball head and stem can be developed in one piece or two pieces. With a two-piece development the stem and the ball head are separate components that can be connected to each other. The connection can be achieved by positive locking, e.g. click elements, or by positive force locking, e.g. a threaded connection. Stem and ball head are preferably connected via a conical attachment on the stem and a corresponding conical recess in the ball head. The arrangement can also be reversed and the conical attachment can be developed on the ball head and the conical recess in the stem. On the end of the stem facing the ball head there is preferably provided a support surface that widens in the form of a disk and which rests on the resection plane.

The underside of the ball head or of the support surface is closed and can be smooth, slightly convex or slightly concave. In particular when the underside is developed convex, the result is a very homogeneous distribution of the load originating from the joint over the corticalis and the spongiosa.

With the endoprosthesis according to the invention there is an optimum reproduction of the centre of rotation as a result of which a force transmission is achieved that largely corresponds to the anatomical starting situation. In the case of conventional long-stemmed systems on the other hand the stem anchored in intramedullary manner presets a certain area for the centre of rotation of the ball head. Thus often the anatomic variation of the seat of the ball head with its centre of rotation cannot be matched, with a resulting force transmission that deviates from the anatomical starting situation. Even with very costly, settable prostheses this cannot be avoided, or only by means of a complicated operating technique.

Starting from the underside of the ball head or of the support disk the stem tapers, preferably conically or over a radius, in the direction of the stem tip. The region directly adjacent to the underside of the ball head or of the support disk can also be developed cylindrical.

For rotation resistance, the stem preferably has one or more ribs that generally extend radially around the longitudinal direction of the stem. For rotation resistance, the stem can also be developed prismatic with smooth external surfaces.

The advantages achievable with the invention are in particular that the loads starting from the joint are transmitted homogeneously to the corticalis and the spongiosa with the result that here there is no bone degradation or reformation. As a result of the conicity of the stem with an angle of up to 30°, preferably approximately 20°, a secure seat of the stem in the spongiosa is achieved by clamping, which is called “press fit” in the present field.

The stem axis lies preferably on the same axis that runs through the ball centre perpendicular to the ball-closure surface or underside. The support and force transmission is therefore achieved via the cortical ring in the resection plane and also via press fit into the spongiosa or into the cement plug during cementation. The head rests flat, via the ball-closure surface or the support surface of the stem, on the resection plane, even after inaccurate resection and stem seat opening and also retrotorsion and inclination fluctuations, and thus ensures a homogeneous force transmission close to the original preoperative situation. Thus the risk of an osteolysis is reduced, in particular compared with surface replacement prostheses with cap-shaped balls hollowed out inside.

In the case of the implantation of the endoprosthesis according to the invention in the shoulder, the joint ball of the humerus is completely resected. As a result the free space required to supply or implant the glenoid is created.

The reproduction of the mediodorsal offset of the ball head to the stem can be ensured with a precise instrumentation when opening the stem channel.

To allow the bone tissue to grow in, one or more recesses can be provided in any arrangement in the stem, e.g. axially, radially or diagonally running mill cuttings, bores or slots. Several pins on an outer ring around the stem axis can contribute to the anchoring and absorption of shear forces and for rotation resistance. The underside of the ball head or support surface is preferably structured, e.g. provided with grooves or coated porous.

The stem part can be made from titanium or cobalt-chromium alloys and the joint head can be made from a cobalt-chromium alloy or ceramic. Stem part and/or joint head can also be made from non-reinforced or fibre-reinforced plastic.

In the case of the endoprosthetic care of joints, so-called cup prostheses are used as surface replacement for a weakened joint head. The ball head is still generally relatively well maintained with the result that the endoprosthesis is in principle a hemispherical cap that is fitted onto the joint head. On the other hand if the joint head is largely destroyed, a long-stemmed prosthesis is usually used. The joint endoprosthesis according to the invention is in many cases an alternative to such long-stemmed prostheses, but also to the mentioned short-stemmed prostheses with cap-shaped balls, and represents a concept not previously realized with such consistency in shoulder endoprosthetics. Only in the case of joint fractures can a long-stemmed prosthesis often not be avoided.

### BRIEF DESCRIPTION OF THE DRAWINGS

Embodiments of the invention are explained in the following using the drawing. There are shown in:

FIGS. 1 to 3 a one-piece joint endoprosthesis seen from below and from the side or in a three-dimensional representation;

FIGS. 4 to 6 a second embodiment of a one-piece joint endoprosthesis seen from below, from the side or in perspective;

FIGS. 7 to 10 an embodiment of a two-piece joint endoprosthesis seen from below, from the side in disassembled state, from the side in assembled state or in a perspective representation;

FIGS. 11 to 14 a second embodiment of a two-piece joint endoprosthesis seen from below, from the side and exploded, from the side in assembled state or in perspective;

FIGS. 15 to 18 the stem of a two-piece joint endoprosthesis seen from below, from the side, in longitudinal section or in perspective;

FIGS. 19 and 20 two versions of the joint endoprosthesis in section;

FIGS. 21 and 22 a version of the joint endoprosthesis with a stem inclined at an angle;

FIG. 23 a version with grooved underside of the ball head; and

FIG. 24 in section, the implanted joint endoprosthesis.

### IMPLEMENTATION OF INVENTION

The stem 10 is fluted with four grooves 11 between each of which ribs 12 stand. The outside edges of the ribs 12 are slightly concavely curved. The ribs 12 taper downwards and end in a rounded tip 13. The outside edge of each rib 12 runs at an angle of approximately 10° to the longitudinal axis of the stem 10. The surface of the grooves 11 is on the other hand axially aligned.

The ball head 20 has an arched top side 21 and a flat underside 22 and thus overall has the shape of a sphere section, the sphere section being less than a hemisphere. The angle at centre a is less than 170°.

The transverse diameter of the ball head 20 can be for example 43 mm and the length of the stem 10 on the other hand only 25 mm. Thanks to the fluting of the stem the endoprosthesis sits rotation-resistant. The tapering of the ribs 12 towards the blunt tip 13 produces a kind of press fit of the prosthesis in the spongiosa. The underside 22 of the ball head 20 rests flat on the resection plane.

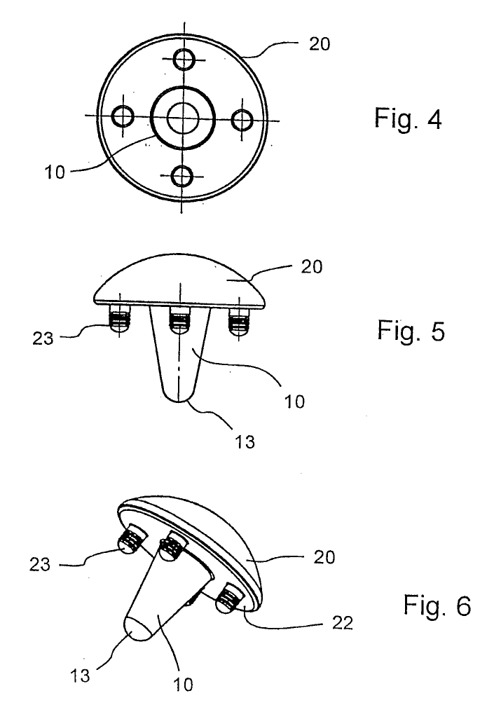

FIGS. 4 to 6 likewise show an embodiment of a one-piece joint endoprosthesis. The surface of the stem 10 is conical and smooth. The tip 13 is again rounded. The ball head 20 again has the shape of a sphere section. Four pins 23 that secure the endoprosthesis against shear forces and rotation project downwards from the underside 22. The pins 23 are approximately 5 to 20 mm long, have a diameter of approximately 4 to 10 mm and are provided on their surface with grooves or threads and/or coated, whereby a better anchoring of the pins in the corresponding bores of the spongiosa is achieved.

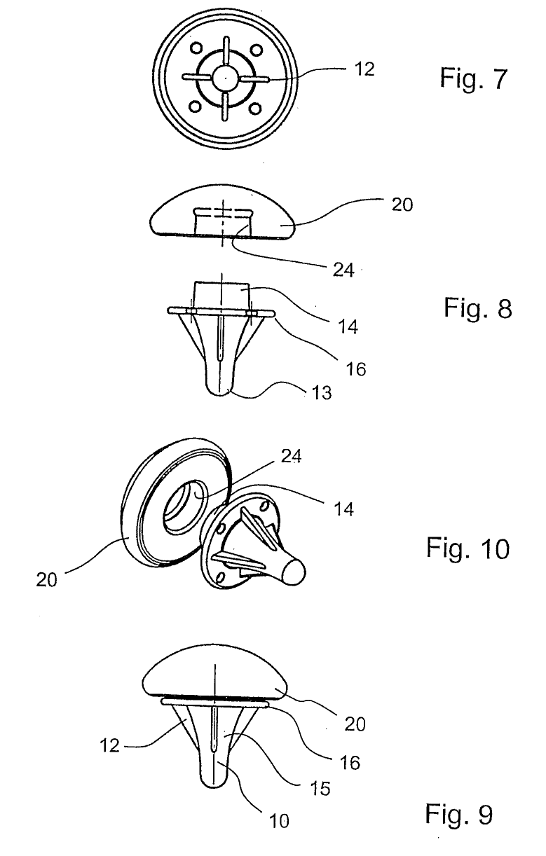

FIGS. 7 to 10 show an embodiment of a two-piece joint endoprosthesis, the stem 10 and the ball head 20 being developed as separate elements that can be joined by means of a cone 14 on the upper end of the stem 10 and a corresponding conical recess 24 in the underside of the ball head 20. The stem 10 is composed of an axial pin 15 that tapers with a concave surface towards the blunt tip 13. A support disk 16 lying across the longitudinal axis is developed between the cone 14 and the pin 15. Four ribs 12, the outside edges of which meet at an angle of approximately 20 to 70°, preferably approximately 60°, are provided, regularly distributed over the periphery, between the support disk 16 and the pin 15.

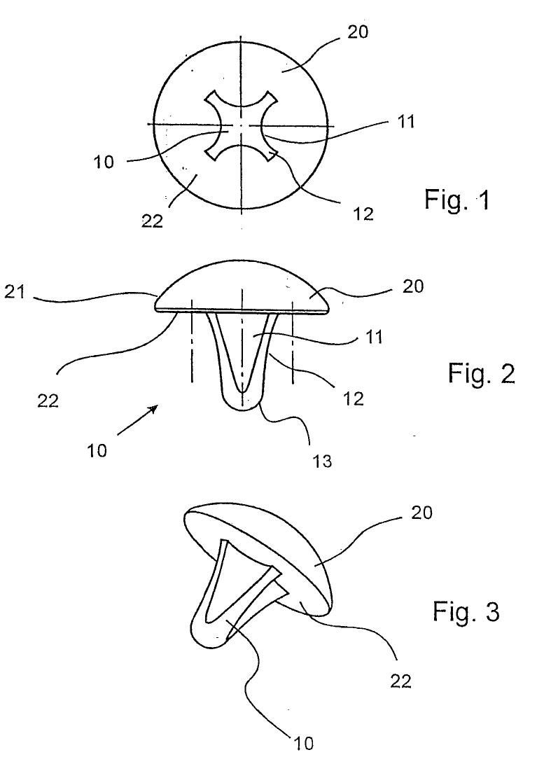

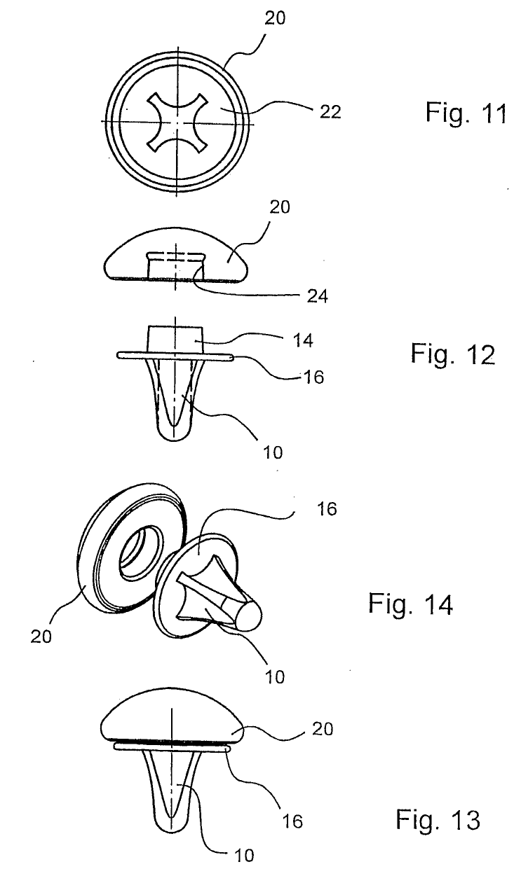

FIGS. 11 to 13 show a further embodiment of a two-piece joint endoprosthesis. The stem 10 is fluted similar to that of the embodiment of FIGS. 1 to 3. The connection between the stem 10 and the ball head is, as with the embodiment of FIGS. 7 to 10, created by a cone 14 on the upper end of the stem 10 and a correspondingly conical recess and a ball head 20. A support disk 16 is also provided here between the stem 10 and the cone 14.

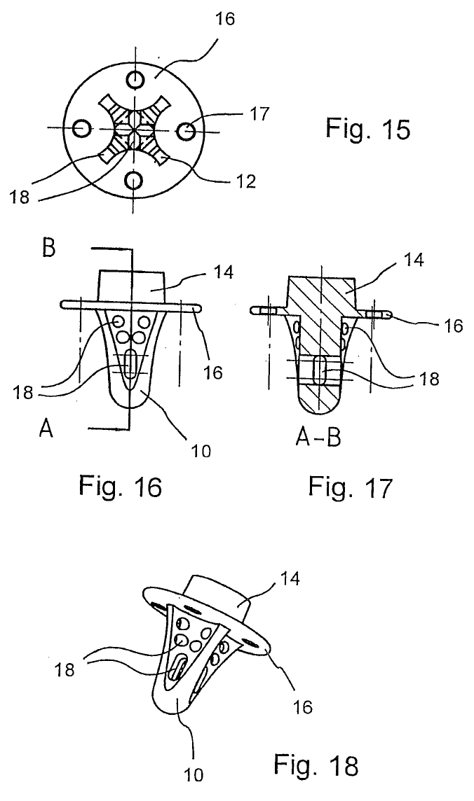

FIGS. 15 to 18 show the stem 10 of a two-piece joint endoprosthesis similar to that of FIGS. 11 to 14, the stem 10 being provided with openings 18 of circular or oblong cross-section. The openings 18 allow the bone substance to grow in.

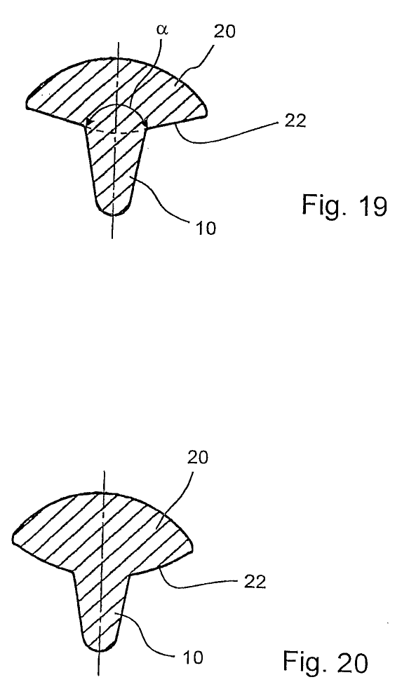

FIGS. 19 and 20 show in section joint endoprostheses with a ball head 20 and a short stem 10, the underside 22 of the ball head 20 being convex and flat-conical downwards (FIG. 19) or arched towards the bottom or the top (FIG. 20). The ball head 20 hereby has a lens shape overall. The convex shape of the underside 22 results in a particularly homogeneous transmission of the forces starting from the joint to the corticalis and the spongiosa.

FIGS. 21 and 22 show one-piece joint endoprostheses similar to those of FIGS. 1 to 3, the stem 10 however being inclined at an angle of approximately 10° to the axis of he ball head 20. As with the version of FIGS. 7 to 9, the stem is provided with radial ribs 12. The inclination of the stem 10 at an angle to the ball head 20 can sometimes be expedient to prevent the stem touching the corticalis lying opposite the resection plane.

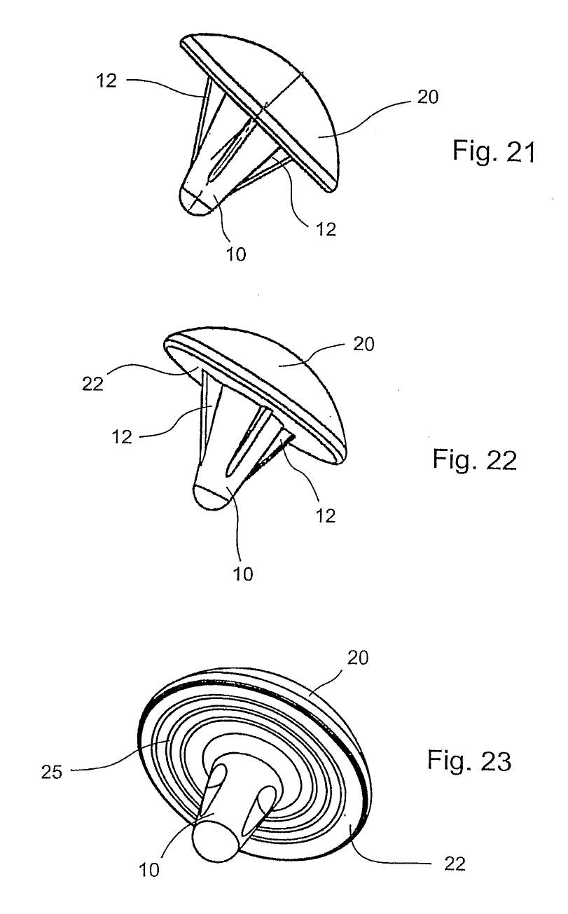

FIG. 23 shows obliquely from below an endoprosthesis in which the underside of the ball head 20 has a spiral groove 25. The groove 25 makes possible a better growing in of the bone.

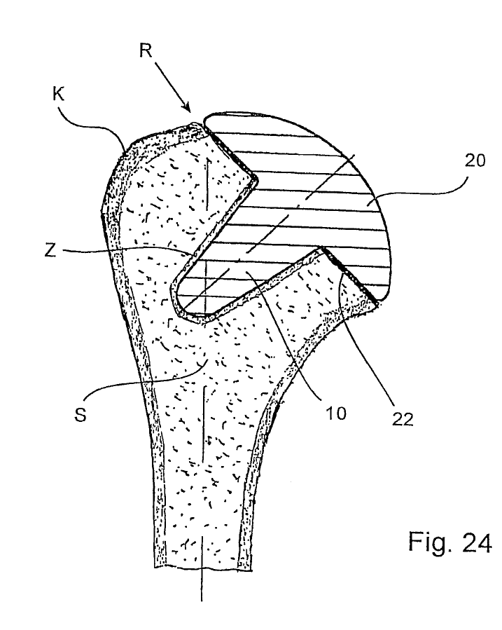

FIG. 24 shows in section the implanted joint endoprosthesis. The flat underside 22 of the ball head 20 sits plane on the resection plane R and the stem extends into the spongiosa S, its tip maintaining an adequate distance from the corticalis K. In FIG. 24 between the underside 22 of the ball head 20 and the resection plane and between the stem 10 and the spongiosa S a narrow region 2 is recognizable that can be a cement plug or a coating of the endoprosthesis. Such a cement plug or such a coating can be present. They are not necessary in every case however.

What is claimed is:

1. A hip- or shoulder-joint endoprosthesis comprising:
a stem (10), which tapers with an overall aperture angle in the range of 5 to 30°; and
a ball head (20), which has an arched top side (21) with the shape of a sphere section of less than a hemisphere and a closed underside (22);
wherein the stem (10) and the ball head (20) each has a longitudinal axis, the axis of the ball head (20) runs through the ball centre and perpendicular to the underside (22), and the longitudinal axis of the stem (10) forms an angle of at most 30° with the axis of the ball head (20); and
wherein the length of the stem (10) is less than the diameter of the ball head (20).

2. The endoprosthesis according to claim 1, wherein the longitudinal axis of the stem (10) coincides with the axis of the ball head (20).

3. The endoprosthesis according to claim 1, wherein the stem (10) tapers with an overall aperture angle of approximately 20°.

4. The endoprosthesis according to claim 1, wherein the endoprosthesis is two-piece, the stem (10) and the ball head (20) being separate parts that can be connected to each other.

5. The endoprosthesis according to claim 4, wherein the stem (10) and the ball head (20) are connected to each other by one of positive locking or positive force locking.

6. The endoprosthesis according to claim 4, wherein the connection of stem (10) and ball head (20) takes place via a conical attachment (14) on the stem (10) and a corresponding conical recess (24) in the ball head (20) or vice versa.

7. The endoprosthesis according to claim 4, wherein at its end facing the ball head (20) the stem (10) widens to form a support disk (16) against which the ball head (20) rests.

8. The endoprosthesis according to claim 1, wherein the stem (10), starting from the underside (22) of the ball head (20) or support disk (16), tapers conically or over a radius in the direction of the stem tip (13).

9. The endoprosthesis according to claim 1, wherein the stem (10), starting from the underside of the ball head (20) or support disk (16) is developed cylindrical.

10. The endoprosthesis according to claim 1, wherein one or more ribs (12) are developed on the stem (10).

11. The endoprosthesis according to claim 1, wherein the stem (10) has at least one recess or opening (18), selected from the group consisting of axially, radially or diagonally running mill cuttings, bores or slots.

12. The endoprosthesis according to claim 1, wherein the underside (22) of the ball head (20) or of the support disk (16) is flat.

13. The endoprosthesis according to claim 1, wherein the underside (22) of the ball head (20) or of the support disk (16) is flat-conical, arched-convex or arched-concave.
